(19) United States
(12) Patent Application Publication
(10) Pub. No.: US 2008/0176908 A1
(43) Pub. Date: Jul. 24, 2008

(54) Method of using squalene monooxygenase inhibitors to treat acne

(76) Inventor: Weylan R. McAnally, Red Oak, TX (US)

Correspondence Address: Christopher J. McDonald;HOFFMAN, WASSON & GITLER, P.C., Suite 522, 2461 South Clark Street, Arlington, VA, 22202, US

(21) Appl. No.: 11/654,681

(22) Filed: Jan. 18, 2007

(51) Int. Cl. A61K 31/44; A61K 31/135; A61K 31/18

(52) U.S. Cl. 514/350; 514/599; 514/649; 514/671; 514/740

(57) ABSTRACT

A method for the treatment of acne comprising the administration, to a patient afflicted therewith, of a effective amount of a squalene monooxygenase inhibitor such as tolnaftate, naftifine, terbinafine, butenafine or ciclopirox. An advantage of the present invention relates to the surprisingly speedy onset of effectiveness in relieving acne symptoms. The compound may be administered orally or topically.

### FIELD OF THE INVENTION

This invention relates to a pharmaceutical composition useful for the treatment of acne.

### BACKGROUND OF THE INVENTION

Acne is a common inflammatory disease which is very common at puberty but may continue into adulthood. It occurs in skin areas where sebaceous glands are largest, most numerous and most active. In milder forms, acne is a superficial disorder which can be treated adequately by ordinary skin hygiene. However, pilosebaceous follicles occur and result in the formation of pustules, infected cysts and, in extreme cases, canalizing inflamed and infected sacs, which may become extensive and leave permanent, disfiguring scars.

Therapeutic methods for treating acne include systemic and topical administration of anti-acne agents such as antibiotics or derivatives of Vitamin A acid. In all but the most severe cases, systemic treatment of acne is not desirable due to side effects. However, systemic methods have been used extensively to treat acne due to the unavailability of a topical formulation which possesses a level of therapeutic effectiveness desirable to relieve symptoms of acne.

Squalene monooxygenase (formerly squalene epoxidase) catalyzes the second committed (and likely rate-limiting) step in cholesterol biosynthesis from farnesyl pyrophosphate, making it an attractive pharmacotherapeutic target in the management of hypercholesterolemia and resultant cardiovascular disease. In neural tissue squalene monooxygenase plays an essential role in the synthesis of cholesterol necessary for myelin membranes, and has been identified as the site of inhibition by tellurium-containing compounds that cause a peripheral neuropathy. But, despite its pivotal role in cholesterol biosynthesis, remarkably little is known about this enzyme, especially in man.

It is an object of the invention to use squalene monooxygenase inhibitors in the treatment of acne.

It is another object of the invention to use squalene monooxygenase inhibitors in a topical solution.

It is still another object of the invention to provide a quick acting acne treatment.

These and other objects of the invention will be apparent to one of ordinary skill in the art after reading the disclosure of invention.

### SUMMARY OF THE INVENTION

This invention relates to a method for the treatment of acne comprising the administration, to a patient afflicted therewith, of a effective amount of a squalene monooxygenase inhibitor such as tolnaftate, naftifine, terbinafine, butenafine or ciclopirox. An advantage of the present invention relates to the surprisingly speedy onset of effectiveness in relieving acne symptoms. The compound may be administered orally or topically.

### DETAILED DESCRIPTION OF THE INVENTION

The term “squalene monooxygenase inhibitor” or “SMI” used herein to refer to a class of substances from the thiocarbamate, allylamine, benzylamine or hydroxypryridone chemical structural classes.

An exemplary thiocarbamate is tolnaftate and its pharmaceutically acceptable salts, prodrugs and esters such as its hydrochloride and phosphates. Tolnaftate is known by the IUPAC name of O-(2-naphthyl)methyl(3-methylphenyl)thiocarbamate. Tolnaftate is disclosed in U.S. Pat. No. 3,334,126 (Miyazaki) as being useful as a fungicide for fungal infections on the human skin. This patent is incorporated herein by reference.

Exemplary allylamines include terbinafine and naftifine and their pharmaceutically acceptable salts, prodrugs and esters such as their hydrochlorides and phosphates. Terbinafine is known by the IUPAC name N,6,6-trimethyl-N-(naphthalene-1-ylmethyl)hept-2-en-4-yn-1-amine and is disclosed in U.S. Pat. No. 4,755,534 (Stuetz) as possessing chemotherapeutic activity, specifically as antimycotic agents when administered orally. Naftifine is known by the IUPAC name (E)-N-cinnamyl-N-methyl-1-naphthalenemethylamine and is disclosed in U.S. Pat. No. 4,282,251 (Berney) as possessing chemotherapeutic activity, specifically as antimycotic agents when administered orally. This patent is incorporated herein by reference.

An exemplary benzylamine is butenafine and its pharmaceutically acceptable salts, prodrugs and esters such as hydrochloride and phosphates. Butenafine is known by the IUPAC name N-methyl-1-naphthalen-1-yl-{(4-tert-butylphenyl)methyl}methenamine. Butenafine is disclosed in U.S. Pat. No. 5,021,458 (Maeda et al.) as an antifungal compound in the form of liquid preparations, ointment, cream and the like at a concentration of 0.01 to 5%. This patent is incorporated herein by reference.

An exemplary hydroxypryridone is ciclopirox and its pharmaceutically acceptable salts, esters, prodrugs and olamine derivatives. Ciclopirox is known by the IUPAC name 6-cylcohexyl-1-hydroxy-4-methyl-pyridin-2-one. Ciclopirox is disclosed in U.S. Pat. No. 7,026,337 (Bohn et al.) as a topically applied antimycotic preparation suitable for the treatment of prophylaxis of dermatomycoses. This patent is incorporated herein by reference.

In the treatment of acne, a topical solution containing a squalene monooxygenase inhibitor is applied to an affected area, once or twice daily or as needed. The topical solution may be a gel, spray, lotion, wash, shampoo, liquid, cream or any other suitable medium for topical solutions. The solution has an effective amount of squalene monooxygenase inhibitor. The amount of squalene monooxygenase inhibitor may be 0.01 to 50 wt. percent.

The compound may also be provides in tablet, pill, liquid or other acceptable medium for oral administration. The compound can also be combined with other acne treatments such as benzoyl peroxide, clindamycin, erythromycin, sulfur, sodium sulfacetamide, tretinoin, adapalene. A tablet of 250 milligrams, taken once a day, would be sufficient but the dosage can be tailored for each individual patient. The medication may also be ‘pulse’ dosed, with one tablet a day for a week, then skip three weeks and repeated.

Although the exact mechanism for relief of acne symptoms by squalene monooxygenase inhibitors is unknown, the following mechanism is a theory:

Squalene is a 30-carbon linear isoprenoid compound structurally similar to beta-carotene. It is primarily known for its key role as an intermediate in cholesterol production. Squalene monooxygenase catalyzes the insertion of an oxygen atom across a carbon-carbon double bond to form an epoxide. Squalene is distributed ubiquitously in human tissues with the greatest concentration in the skin, where it has a continual presence of 10 to 15 percent.

Squalene is not very susceptible to peroxidation and thus functions in the skin as a quencher of singlet oxygen, protecting human skin surface from lipid peroxidation due to UV radiation. While vital to cholesterol synthesis, from a dermatological standpoint, oxidized squalene has been described a cytotoxic, irritant and strongly comedogenic. Peroxidated squalene induces the production of inflammatory mediators leading to increased lipoxygenase activity and increased inflammation. Oxidized squalene appears to produce micro-aerophilic conditions in the skin and sets up and ecological niche for anaerobic flora leading to bacterial colonization. Bacterial colonization is a secondary effect of comedogenesis induced by formation of squalene oxides.

Squalene monooxygenase inhibitors block the action of squalene monooxygenase in the skin and thus the production of 2, 3 oxidosqualene is reduced. Since 2, 3 oxidosqualene is pro-inflammatory, inflammation and redness of acne lesions are reduced. Ciclopirox, naftifine and terbinafine have been shown to have greater anti-inflammatory ability than 2.5% hydrocortisone. Due to their similar mechanism of action, it can be assumed that butenafine and tolnaftate would have similar anti-inflammatory action. Although not specifically found in the literature, I believe ciclopirox to be an SMI. The major pathway of action for ciclopirox involves chelation of polyvalent cations such as Fe3+. Inhibition of these cations results in inhibition of metal dependent enzymes such as squalene monooxygenase. Squalene monooxygenase is dependent on NADPH cytochrome P450 reductase to donate an electron to the reaction. Ciclopirox chelates the ferric component of NADPH cytochrome p450 reductase thus inhibiting the electron transfer to squlane monooxygenase. Squalene monooxygenase is thus inhibited from oxidizing squalene to 2, 3 oxidosqualane.

The mechanisms by which squalene monooxygenase inhibitors are effective in treating acne are as follows:
1. SMI compounds prevent the formation of pro-inflammatory 2, 3 oxidosqualene in the skin.
2. SMI compounds increase the level of oxygen quenching, anti-inflammatory squalene in the skin.
3. Increased levels of squalene and decreased levels of oxidosqualene in the skin prevent micro-aerophilic conditions that promote anaerobic bacteria colonization. This colonization would lead to comedogenesis and acne formation.
4. By increasing squalene levels and decreasing oxidosqualene levels, SMI compounds can deter or even prevent future acne lesion formation.

These mechanisms lead to decreased redness and inflammation in existing acne lesions resulting in expedited resolution of acne symptoms. In addition, these mechanisms aid in prevention of future acne lesion formation.

While the invention has been disclosed with reference to preferred embodiments, variations and modifications would be apparent to one of ordinary skill in the art. The invention encompasses such variations and modifications.

I claim:

1. A method for treating acne, comprising
applying a topical solution to an affected area, the solution containing an effective amount of a squalene monooxygenase inhibitor.

2. The method of claim 1, wherein said topical solution contains 0.01-50 wt. percent of squalene monooxygenase inhibitor.

3. The method of claim 1, wherein said squalene monooxygenase inhibitor is chosen from the group consisting of thiocarbamate, allylamine, benzylamine and hydroxypryridone.

4. The method of claim 1, wherein said squalene monooxygenase inhibitor is tolnaftate and its pharmaceutically acceptable salts, prodrugs and esters.

5. The method of claim 1, wherein said squalene monooxygenase inhibitor is terbinafine and its pharmaceutically acceptable salts, prodrugs and esters.

6. The method of claim 1, wherein said squalene monooxygenase inhibitor is naftifine and its pharmaceutically acceptable salts, prodrugs and esters.

7. The method of claim 1, wherein said squalene monooxygenase inhibitor is butenafine and its pharmaceutically acceptable salts, prodrugs and esters.

8. The method of claim 1, wherein said squalene monooxygenase inhibitor is ciclopirox and its pharmaceutically acceptable salts, esters, prodrugs and olamine derivatives.

9. The method of claim 1, wherein said topical solution is a gel, spray, lotion, wash, shampoo, liquid or cream.

10. A method for treating acne, comprising administering a compound containing an effective amount of a squalene monooxygenase inhibitor to a person for the relief of acne.

11. The method of claim 10, wherein said topical solution contains 0.01-50 wt. percent of squalene monooxygenase inhibitor.

12. The method of claim 10, wherein said squalene monooxygenase inhibitor is chosen from the group consisting of thiocarbamate, allylamine, benzylamine and hydroxypryridone.

13. The method of claim 10, wherein said squalene monooxygenase inhibitor is tolnaftate and its pharmaceutically acceptable salts, prodrugs and esters.

14. The method of claim 10, wherein said squalene monooxygenase inhibitor is terbinafine and its pharmaceutically acceptable salts, prodrugs and esters.

15. The method of claim 10, wherein said squalene monooxygenase inhibitor is naftifine and its pharmaceutically acceptable salts, prodrugs and esters.

16. The method of claim 10, wherein said squalene monooxygenase inhibitor is butenafine and its pharmaceutically acceptable salts, prodrugs and esters.

17. The method of claim 10, wherein said squalene monooxygenase inhibitor is ciclopirox and its pharmaceutically acceptable salts, esters, prodrugs and olamine derivatives.

18. The method of claim 10, wherein said compound is administered orally.
